(19) United States
(12) Patent Application Publication
(10) Pub. No.: US 2026/0327680 A1
(43) Pub. Date: Oct. 22, 2026

(54) DEVICE AND SYSTEM FOR COLLECTING AEROSOL PARTICLES AND PREPARING THE SAMPLE FOR ANALYSIS

(71) Applicant: AVELO AG, Schlieren (CH)

(72) Inventors: Tobias Broger, Oberengstringen (CH); Patricia Risch, Dietikon (CH)

(21) Appl. No.: 19/111,191

(22) PCT Filed: Oct. 26, 2023
(86) PCT No.: PCT/EP2023/079951

§ 371 Date: Mar. 12, 2025

(30) Foreign Application Priority Data
Oct. 27, 2022 (CH) CH001271/2022

(51) Int. Cl. C12Q 1/6806; A61B 10/00; B01D 46/00; B01D 46/10; G01N 1/40; G01N 33/497

(52) U.S. Cl. CPC C12Q 1/6806; A61B 10/00; B01D 46/0005; B01D 46/0035; B01D 46/10; G01N 1/4077; G01N 33/497; A61B 2010/0087; B01D 2265/029; B01D 2265/06; B01D 2275/202; G01N 2001/4088

(57) ABSTRACT

A device and system for collecting aerosol particles contained in an aerosol sample and preparing the sample for analysis. The aerosol can be breath of a human or animal or an environmental sample. In at least some embodiments, the present invention provides such a device and system to collect, preserve, concentrate aerosol particles from aerosols and release the aerosol particles into aqueous solution for the subsequent analysis with immunoassays and molecular assays for detection of pathogens. The sampling device comprises a filter assembly, wherein the filter assembly includes an aqueous solution dissolvable element and a filter element installed in a housing of the sampling device in the flow path of the aerosol samples that sorbs and/or filters the aerosol particles. After the collection, upon releasably connecting an elution device to introducing an aqueous solution into the housing, the aqueous solution dissolvable element is dissolved and the filter element is submerged in the aqueous solution to retrieve the aerosol particles in a highly efficient way.

### TECHNICAL FIELD

The present invention relates to a device and system for collecting aerosol particles and preparing the sample for analysis and detection of biomarkers. Such particles are suspended in air in the form of an aerosol and may carry pathogens or biomarkers. The source of the aerosol can be breath of a human or animal or the environment. Such a device and system is configured for the improved collection, preservation, preparation, concentration and rapid and complete release of pathogens and biomarkers for the subsequent analysis with immunoassays or molecular assays for the detection of such pathogens and biomarkers.

### Purpose of the Invention and State of the Art

Up to 20% of all primary care consultations worldwide are due to respiratory infections. They result in 4 million deaths per year-almost all due to lower respiratory tract infections (LRTIs) causing pneumonia (Vos et al. Lancet 2020; 396 (1082): 1204). On the one hand, bacterial infections require timely initiation of antibiotic treatment whereas, on the other hand, viral infections, such as influenza require treatment with antivirals if available.

Therefore, there is a strong need within the in-vitro diagnostics (IVD) industry to deliver improved rapid diagnostic solutions which detect respiratory tract infections in order to break the chain of transmission, initiate the right and timely treatment and avoid prescription of unnecessary antibiotics to fight antibiotic drug resistance. Rapid and simple sample collection and testing relies on easily available sample. High priority target product profiles (TPP's) that describe the unmet diagnostic needs were developed by the World Health Organization (WHO) and other stakeholders and include (a) a TPP for a point-of-care test for community-acquired lower respiratory tract infection (Gal et al. PLOS One. 2018; 13 (8): e0200531) and (b) a TPP for a rapid non-sputum based test for detecting Tuberculosis (TB) (World Health Organization, High-priority target product profiles for new tuberculosis diagnostics: report of a consensus meeting, 2014 and Denkinger. JID. 2015; 211 (Suppl2)).

There are numerous prior art examples describing the use of swabs, sputum, saliva, and bronchoalveolar lavage (BAL) for the diagnosis and detection of respiratory tract infections. These specimens limit the diagnosis of respiratory infectious diseases, particularly lower respiratory tract infections (LRTIs) as follows: (a) Nasopharyngeal or oropharyngeal swabs cause discomfort to the patient, require trained staff and miss LRTIs and TB, (b) saliva and nasal swabs are potentially interesting for self-testing or at-home collection but contain no, or only low amounts of bacteria and viruses from the lower respiratory tract, limiting their use to diagnosis of upper respiratory tract infections, (c) sputum is the most common specimen for TB diagnosis but is difficult to obtain and the inhomogeneous sample matrix requires complex and costly sample preparation, (d) bronchoalveolar lavage (BAL) is the gold standard to diagnose LRTIs but requires special equipment (which is not available at the point-of-care), is highly invasive and may lead to complications. In sum, specimens from the upper respiratory tract might miss LRTIs and specimens from the lower respiratory tract are hard to obtain or require invasive procedures not feasible at the point-of-care.

To overcome these limitations of existing specimens, particularly in the diagnosis of lower respiratory tract infections, the use of breath samples from humans and/or animals is desirable.

There are numerous prior art examples that describe the collection and detection of volatile organic compounds (VOCs) in breath on sensor arrays such as electronic noses or mass spectrometry (U.S. Pat. No. 10,413,215, WO2017187141). However, the molecular entities of the detected VOCs and their origin are usually not defined and thus their relevance for a particular disease is unclear or even biologically implausible which led to low specificities, making VOC based systems inappropriate for clinical diagnosis. Further, mass spectrometers remain expensive, are relatively large instruments and therefore most likely not feasible for point-of-care use. Additionally, collected VOCs are not compatible with state-of-the art laboratory diagnostic methods such as polymerase chain reaction (PCR) and immunoassays.

In contrast, it is well understood that human aerosols contain biologically plausible, nonvolatile pathogens, such as entire bacteria, virus, fungi and pathogen biomarkers such as nucleic acids (such as DNA and RNA) or pathogen antigens. Aerosols are a suspension of fine particles, particulate matter, “droplet nuclei” or liquid droplets (subsequently called “aerosol particles”) in air. Physically speaking, an aerosol is a heterogeneous mixture of particles together with the gas or gas mixture surrounding them. Exhaled aerosol particles occur in multiple size modes that are associated with different generation sites and production mechanisms in the respiratory tract (Wang et al. Science. 2021; 373 (6558)). Aerosol particles are typically ≤100 μm in diameter and may contain infectious bacteria and viruses and studies have shown that pathogens are enriched in small aerosol particles, typically ≤5 μm (Gralton et al. J Med Virol. 2013; 85 (12): 2151, Fennelly et al. Am J Respir Crit Care Med. 2004; 169 (5): 604). Aerosols that contain biological material such as cells like bacteria, or virus, biological molecules, by-products of metabolism and cell fragments are often referred to as bioaerosols which origin from a biological source or may affect a biological target. Many studies showed that respiratory infectious diseases including RSV (Kulkarni et al. Am J Respir Crit Care Med. 2016; 194 (3): 308), MERS-COV (Kim et al. Clin Infect Dis. 2016; 63 (3): 363), influenza (Yan et al. Proc Natl Acad Sci. 2018; 115 (5): 1081), and SARS-COV-2 (Liu et al. Nature. 2020; 582 (7813): 557) are spread by particulates and aerosols (droplet and droplet nuclei) from coughing, sneezing, breathing, and talking. For Tuberculosis, the pioneering experiments of Riley and Wells (Am J Hyg. 1959; 70:185) more than 60 years ago proved airborne transmission by demonstrating that guinea pigs developed TB upon breathing air from remote ward housing TB patients. Since then, the presence of Mycobacterium tuberculosis (Mtb) and related biomarkers in aerosols has been well described in multiple research studies summarized by Fennelly et al. (Chest. 2020; 157 (3): 540).

Current sample collection and concentration devices for pathogen biomarkers from human breath aerosols have many desirable attributes but are inefficient and thus insufficiently sensitive, too complex, contain movable parts, have a high breathing resistance and/or do not teach a way for rapid and instrument-free in situ elution or extraction of pathogens or pathogen biomarkers with high recovery. More efficient yet simpler collection of pathogen biomarkers from human breath with subsequent rapid in situ elution of biomarkers in a suitable volume of 0.5 to 5 ml aqueous liquid that can be used as a sample for detection with immunoassays or molecular assays would clearly be desirable.

The background art fails to teach or suggest the reproducible collection and elution of biomarkers in aerosols. Further the background art fails to teach a device or system that combines efficient aerosol particle collection on filters paired with simple and complete in situ extraction or elution of said aerosol particles in a suitable volume of liquid without moving, removing and/or dipping said filter.

There are prior art examples that describe the collection of specific biomarkers in aerosols with filter systems, all of which fail to meet medical standards due to low sensitivity, low filter efficiency, incomplete elution, complexity, incompatibility with existing diagnostic assays, and/or their instrument requirements.

US2015/0377748 describes a breath analysis system with a filter assembly that is movable between a breath capture position and an analysis position that does not allow for in situ elution and extraction of the collected breath markers. WO 2022/079195 relates to a collection device for collection of particles and presentation of collected particles for analysis. Both systems describe on-board analysis and do not describe a releasably engageable tube for in situ elution and extraction of the collected markers from the collection element to retrieve a sample for biobanking, transportation and/or subsequent processing and analysis with state-of-the art laboratory tests on state-of-the-art laboratory analyzers.

WO 2021/242907 relates to an insert for a wearable mask worn by an individual over the mouth and nose, whereas the test substrate is located inside the mask, and whereas the test substrate is removed from the mask and inserted into a vial for further testing. This requires manual handling of the substrate and poses a high risk of contamination of the sample with biomarkers and pathogens from the environment and at the same time puts the user at risk when manually handling infectious material on the substrate. Inefficiency and resulting low sensitivity is also a limitation in another prior art examples due to the low collection and concentration efficiency (EP 1 377 815).

Another limitation is the insufficient, incomplete and time-consuming release of the collected specific biomarkers for subsequent detection (US 2020/0300876). Limitations of other, prior art examples for biological air and aerosol particles sampling are their requirement for active pumping which makes the instruments complex and unsuitable for human breath sampling with tidal breathing or exhalation where low breathing resistance is needed (US 2013/0273520). Further, prior art examples do not describe an instrument-free combination of collection and elution.

WO 2012/024407 describes an aerosol collection system that uses a nanofiber mat with a plurality of electrospun nanofibers formed as a filter to collect aerosols. A pump is used to entrain air-borne particles in a gas stream.

WO 2013/132085 relates to a portable sampling device for aerosols. An electrostatic filter membrane is used to collect aerosols from exhaled breath of a subject. To filter out contaminants, such as saliva, mucus and large particles, baffle plates inside the sampling device are provided to obtain a non-straight gas flow through the device. After use, the device is sealed and then sent to a laboratory for further sensor based analysis. Solvents are used for extraction.

U.S. Pat. No. 10,080,857 describes a system for breath sample collection and analysis. A subject exhales into a mouthpiece connected to a sample collector. The sample collector uses a nozzle and contains a liquid buffer downstream of the nozzle which collects the analyte. After use, the mouthpiece is disconnected, the sample collector is sealed and then transferred to a diagnostic device which analyses the analyte.

### BRIEF SUMMARY OF THE INVENTION

It is an object of the present invention to eliminate, or at least mitigate the problems associated with prior art devices, systems and methods. In particular, it is an object of the present invention to not only improve the collection of non-volatile respiratory pathogens present in human or animal breath aerosol particles, but also the handling of the sample for the subsequent analysis with immunoassays and molecular assays for the clinical diagnosis of respiratory tract infections in humans.

These and possibly other objects of the invention are solved by the feature combinations of independent claim 1. Preferred or optional features of the invention are indicated in the dependent claims 2-29.

The present invention relates to a device and system for collecting aerosol particles and preparing the sample for analysis. Although reference is made herein to “human breath”, it is contemplated that the present invention would also be suitable for non-human animals. In addition, the present invention may also be used to collect particles from air in a room, for example, in which human beings gathered in order to test the air exhaled by the human beings for pathogens or biomarkers possibly present in the air. In addition, the present invention may also be used to sample aerosol from livestock and the environment.

In at least some embodiments, the present invention provides such a device and system to collect, preserve, concentrate and release pathogen-specific biomarkers from breath aerosol particles for the subsequent analysis with immunoassays and molecular assays for the clinical diagnosis of respiratory tract infections in humans. The sampling device comprises an aqueous solution dissolvable element and a filter element in spatial proximity installed in the housing in the flow path of aerosol. For the purposes of the present disclosure, a dissolvable material is defined as a material of which 4 mg dissolve within less than 15 minutes at ambient temperature (23° C.), ambient pressure (1 atm) and in 1.6 mL water without movement of the water by stirring, shaking and the like.

The sampling device for the collection of aerosol particles contained in an aerosol sample comprises a housing, which comprises a flow inlet for the inflow of an aerosol sample into the housing and a flow outlet for the outflow of the aerosol sample out of the housing. The flow of the aerosol sample from the flow inlet to the flow outlet defines a flow path of the aerosol sample. In other words, the housing defines, respectively delimits, a flow path for the aerosol sample from the flow inlet to the flow outlet. The sampling device may further comprise a filter assembly to collect aerosol particles contained in the aerosol sample. Particularly, the filter assembly includes an aqueous solution dissolvable element and a filter element. The filter assembly is preferably at least partially arranged inside the housing between the flow inlet and the flow outlet and in the flow path of the aerosol sample.

A system for the collection and elution of aerosol particles contained in an aerosol sample according to the invention may comprise a sampling device as described in any of the embodiments herein, and an elution device which is releasably connectable to the housing by means of the engagement means of the sampling device for introducing an aqueous solution into the housing, for dissolving the aqueous solution dissolvable element upon contact with the aqueous solution, and upon dissolution of the aqueous solution dissolvable element, for submerging the filter element into the aqueous solution to thus retrieve the aerosol particles captured on the aqueous solution dissolvable element and the filter element and receiving the eluate.

In some embodiments, both the aqueous solution dissolvable element and the filter element are arranged inside the housing between the flow inlet and the flow outlet and in the flow path of the aerosol sample. Therefore, both the aqueous solution dissolvable element and the filter element are exposed to the aerosol sample when it flows through the flow path.

In some embodiments, the aqueous solution dissolvable element is airtight. As used herein the term “airtight” for an element such as the aqueous solution dissolvable element may preferably mean that air at atmospheric pressure does not pass the airtight element. For example, if a person exhales into the flow path (e.g. via the flow inlet), the exhaled air does not pass through the airtight aqueous solution dissolvable element. In certain embodiments, the airtight element may be configured such that air at a pressure of 1.2 bar and at 23° C. does not pass through the element with a rate higher than 1 mL per hour, in particular higher than 1 mL per day.

In some embodiments, the filter element is air permeable. For example this may mean that if a person exhales into the flow path (e.g. via the flow inlet), the exhaled air passes through the air permeable filter element. In certain embodiments, such an air permeable element allows air at a pressure of 1.2 bar and at 23° C. to pass through the element with a rate of more than 1 mL/h, in particular more than 1 mL/min, more particular more than 1 L/min.

In some embodiments an area of the filter element is greater than an area of the aqueous solution dissolvable element. That is, a total surface area of the filter element may be greater than a total surface area of the aqueous solution dissolvable element. It is understood that the total surface area refers to the area of the full surface of the filter element.

In some embodiments, the filter element is at least partially arranged radially outwardly from the aqueous solution dissolvable element. As understood by the skilled person the term “radially outwardly” refers to a direction along a plane along which the aqueous solution dissolvable element extends. Further, it indicates a maximum distance between the center of the aqueous solution dissolvable element towards the outer peripheral limit of the aqueous solution dissolvable element. Thus, if the aqueous solution dissolvable element has a circular cross section it refers to the radius of the circle. If the aqueous solution dissolvable element has a rectangular cross section it refers to half the diagonal extending from the center to an edge of the rectangle.

In some embodiments, the filter element is ring-shaped and/or circular, and the aqueous solution dissolvable element is geometrically arranged completely inside the filter element. In some embodiments, the aqueous solution dissolvable element is peripherally circumferentially surrounded by the filter element.

In some embodiments, wherein a diameter of the filter assembly is between 5 and 50 mm. The term “diameter” as used herein refers to length of a line extending through the filter assembly center from which two opposing peripheral limits of the filter assembly have the largest distance from each other. Thus, if the filter element has for example a circular cross section it refers to the diameter of the circle. If the filter element has for example a rectangular cross section it refers to the diagonal extending edge to edge the rectangle.

In some embodiments, the filter element and the aqueous solution dissolvable element at least partially overlap.

In some embodiments, the filter element and the aqueous solution dissolvable element are bonded.

In some embodiments, the filter assembly further includes a retainer element which retains the filter element and/or the aqueous solution dissolvable element in place.

In certain embodiments the retainer element is clampingly engageable with a filter holder.

In some embodiments, the sampling device further comprises an engagement means for releasably engaging an elution device with the housing, for introducing an aqueous solution into the housing, for submerging the aqueous solution dissolvable element and the filter element into the aqueous solution, and to thus dissolve the aqueous solution dissolvable element upon contact with an aqueous solution.

In some embodiments, the engagement means includes a thread.

In certain embodiments, the engagement means is an internal thread provided on an inner surface of the housing.

In some embodiments, the engagement means is configured such that a laboratory tube containing an aqueous solution and preferably normally used in preparation for an assay selected from the group consisting of a nucleic acid amplification test (NAAT), a PCR assay, an isothermal amplification assay, a DNA hybridization assay, a CRISPR-based assay, a sequencing assay and an immunoassay, is releasably connectable to the housing. A normally used laboratory tube may for example be a Copan eNAT tube with 12 mm diameter and 80 mm long.

In some embodiments, a thread is provided on the housing upstream of the aqueous solution dissolvable element. In such embodiments the thread may be engageable with a threaded closure to close off the flow inlet of the housing.

In some embodiments, the aqueous solution dissolvable element comprises a polymer selected from the group consisting of polyvinyl alcohol (PVA), chitosan, polyethylene oxide (PEO), pullulan, polyvinylpyrrolidone (PVP), polyvinyl acrylic acid (PVAc), poly methacrylic acid (PMAc), methyl acrylate copolymers, hydroxypropyl methylcellulose phthalate (HPMCP), and a combination thereof.

In some embodiments, the aqueous solution dissolvable element is a membrane, foil or film with a thickness in a range between 0.5 μm to 50 μm.

In some embodiments, the filter element is fully or partially dissolvable in an aqueous solution.

In some embodiments, the filter element comprises fibers. Such fibers increase the surface area and help to collect more aerosol particles.

In some embodiments, the filter element comprises two layers of fibers.

In some embodiments, the fibers have a diameter in the range between 20 nm to 1 μm.

In some embodiments, the filter element further comprises a polymer support mesh having a thickness in a range between 40 μm to 500 μm to support the fibers.

In some embodiments, dissolution of the aqueous solution dissolvable element and/or the filter element in the aqueous solution is triggered by a change in pH-value. That is, in certain embodiments, the aqueous solution dissolvable element is configured such that its dissolution can be triggered by a change of pH-value.

In some embodiments, the aqueous solution dissolvable element and/or filter element contain assay reagents selected from the group consisting of control nucleic acid, primers, probes, nucleotides, enzymes, salts, capture beads and combinations thereof.

In some embodiments, the sampling device is a hand-held sampling device for the collection of aerosol particles present in a human breath bioaerosol.

In some embodiments of the system according to the invention, the elution device is a tube having a closed end and an open end opposite the closed end, and wherein an external thread is provided at the open end of the elution device which is engageable with an internal thread of the housing of the sampling device.

In some embodiments of the system according to the invention, the elution tube has a sample volume in the range of 0.2 ml to 20 ml, and/or a diameter in the range of 8 mm to 30 mm.

In a preferred, non-limiting exemplary embodiment the aqueous solution dissolvable element is a circular foil positioned in the center surrounded by a ring-shaped filter element. However, as is readily appreciated by a person skilled in the art, that other spatially close arrangements of the aqueous solution dissolvable element and the filter element are equally possible within the scope of the inventive concept. During sample collection, an aerosol, defined as particles in air, enters the sampling device. Large particles impact with the airtight dissolvable, centric membrane based on inertial impaction which constitutes a first collection or filter stage. Smaller particles that do not impact with the aqueous solution dissolvable element, travel with the airflow to the ring-shaped air permeable filter element where they get filtered in a second collection or filter stage by the filter element. An advantage of this two-stage design is its high efficiency and high filter capacity over the duration of sample collection where the aqueous solution dissolvable element captures larger particles that could otherwise negatively impact the filter capacity of the filter element and/or lead to an increase of the pressure drop of the filter assembly.

Upon elution, the aqueous solution dissolvable element dissolves in an aqueous solution upon contact which creates a centric opening so that the outer filter element is submerged in said aqueous solution. An advantage of this is that the particles trapped on the aqueous solution dissolvable element are completely released into solution upon dissolution of said aqueous solution dissolvable element. Another advantage is that the particles captured by the ring-shaped filter element are eluted from both sides of the filter element due to the centric opening that is formed after dissolution of the aqueous solution dissolvable element. Taken together this leads to very high elution recoveries of captured particles, pathogens and biomarkers. Another advantage is the spatial closeness of the aqueous solution dissolvable element and the filter element which facilitates elution in a small liquid volume. This permits concentrating of particles, pathogens and/or biomarkers from a relatively large air volume in a small volume of liquid by the sampling device to reach sufficient concentrations for subsequent detection with molecular assays and/or immunoassays.

Non-limiting examples of suitable aqueous solution dissolvable elements include polyvinyl alcohol (PVA), chitosan, polyethylene oxide (PEO), hydroxypropyl methylcellulose phthalate (HPMCP), pullulan, or methyl acrylate copolymers or the like. The aqueous solution dissolvable element may be provided in the form of a film or foil or the like. In a preferred embodiment the aqueous solution dissolvable element is a PVA foil. In another preferred embodiment the aqueous solution dissolvable element is a methyl acrylate copolymer with different acidic or alkaline end groups, which allow for pH-dependent solubility to keep the element stable in breath with pH 7 and dissolve it rapidly at pH above 7.

Non-limiting examples of suitable filter elements include filters based on nonwoven fibers such as nanofiber mats or melt-blown fibers, woven materials, fiberglass, electrostatic filters, filter membranes or the like. The filter can be non-dissolvable, partially or fully dissolvable in aqueous solution. Like for the aqueous solution dissolvable element the dissolution can also be triggered by a change in pH. Non-limiting examples for fiber production include electrospinning, melt blowing, blow spinning, wet spinning, direct drawing, centrifugal spinning, force spinning, touch- and brush-spinning, template synthesis, self-assembly, isolating fibers from plants or wood. In a preferred embodiment the filter element is a two-layer arrangement of two non-dissolvable polymer nanofiber mats on polymer support meshes.

Without wishing to be limited by a single hypothesis, such a device provides a portable, hand-held, highly efficient, point-of-care collection and elution system for pathogen biomarkers like nucleic acids and antigens from human breath aerosol particles that is fully disposable and does not rely on complex instruments.

Furthermore, the elution is preferably done by connecting the sampling device to an elution device such as an elution tube containing an aqueous solution and turning it upside down to dissolve the aqueous solution dissolvable element as an effect of gravitational force. By way of example the aqueous solution is a stabilizing and/or inactivating transport buffer containing TRIS, ethylenediaminetetraacetic acid (EDTA), guanidine thiocyanate, guanidine hydrochloride, HEPES, Universal Transport Medium (UTM), liquid amies transport medium, Tween 20, Triton X100, tris(2-carboxyethyl)phosphine (TCEP), sodium chloride, phosphate-buffered saline, Hank's balanced salt solution, bovine serum albumin, L-cysteine, gelatin, sucrose, glutamic acid, vancomycin, amphotericin B, colistin, phenol red, sodium hydroxide, isopropanol or a combination thereof. This enables preservation of the breath aerosol particles containing the pathogens and pathogen biomarkers for the purpose of storage and/or safe shipment to centralized labs for subsequent clinical diagnosis of respiratory tract infections (e.g. with immunoassays and molecular assays). In one particular embodiment, the elution device is a standard clinical lab tube and therefore highly compatible with standard workflows of centralized labs. This not only speeds up the entire process from the point at which the aerosol sample is taken up to the point at which the sample undergoes clinical analysis, but also requires fewer logistic steps and minimizes the contamination risk of the biological sample. The immediate elution of the pathogen and pathogen biomarkers with a stabilizing aqueous solution right after the collection of the aerosol particles with the sampling device preserves the viability of the pathogen and stabilizes the pathogen biomarkers for transportation. In some embodiments, inactivation of the pathogen for safe transportation is desirable which can be achieved by an inactivating buffer that kills the pathogen but preserves the biomarkers. A non-limiting example is the use of guanidine thiocyanate containing buffer which inactivates the pathogen but preserves nucleic acids for transportation of the sample at ambient temperature. By way of example, the elution device containing the collected aerosol particles is shipped to a centralized laboratory for testing.

According to at least some embodiments, such a collection and elution system preferably includes relatively few parts for economical manufacturing. The sampling device preferably outputs a concentrated pathogen biomarker sample with compatibility with a variety of different assays, analyzers and methods for the detection of respiratory pathogens present anywhere in the respiratory tract, including without limitation the upper respiratory tract and/or the lower respiratory tract. Preferably the sampling device does not require powered equipment for collection and elution. Analyzers and assays used with the present invention can be point-of-care systems or centralized lab systems. Non-limiting examples of assays and detection methods include immunoassays, molecular assays including all nucleic acid amplification tests (NAATs such as PCR, isothermal amplification), DNA hybridization assays, CRISPR-based assays, sequencing assays, or growth assays.

Unless otherwise defined, all technical and scientific terms used herein have the same meaning as commonly understood by one of ordinary skill in the art to which this invention belongs. The materials, methods and examples provided herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-B are exemplary, non-limiting perspective cut-away side elevation views of the sampling device with (A) a large accelerator opening and (B) an alternative, optional embodiment of the present invention having a nozzle arrangement as an accelerator.

FIG. 2A-B are exemplary, non-limiting perspective cut-away side elevation views of the filter assembly illustrating (A) the collection of aerosol particles during aerosol sample collection and (B) the centric opening that forms upon dissolution of the aqueous solution dissolvable element upon elution.

FIG. 3A-C are exemplary, non-limiting perspective cut-away views of a detail of the filter assembly shown in FIG. 2 illustrating (A) the filter element and the aqueous solution dissolvable element (B) the centric opening that forms upon dissolution of the aqueous solution dissolvable element upon contact with aqueous solution upon elution, and (C) an example of a preferred embodiment with two stacked filters.

FIG. 4 is an exemplary, non-limiting perspective cut-away side elevation view of the system illustrating the sampling device, elution device, and the aqueous solution during elution.

FIG. 5 shows an exemplary, non-limiting perspective cut-away side elevation view of the previously described sampling device integrated in a face mask.

FIG. 6A-B show results from limit of detection experiments with the sampling device in combination with PCR.

FIG. 7A-B show aerosol filter efficiency results of the sampling device.

FIG. 8 shows results of breathing resistance measurements from the sampling device.

### DESCRIPTION OF AT LEAST SOME EMBODIMENTS

Specific examples of the disclosure will now be described with reference to the accompanying figures. Although specific configurations and arrangements are discussed, it should be understood that this is done for illustrative purposes only. It will be apparent to a person skilled in the pertinent art that this invention can also be employed in a variety of other applications.

It is noted that references in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

Although the description below refers to a sampling device and an elution device, it may well be understood that the sampling device and elution device can also be in a microfluidic arrangement integrated within a microfluidic assay with a detection system for performing a variety of tests, such as immunoassays, isothermal amplification assay, PCR, DNA hybridization, sequencing etc. In an embodiment, the test cartridge integrates all of the components necessary to perform such tests into a single, disposable package. The test cartridge may be configured to be analyzed by an external detector to read the assay signal which has a connection to a data entry, data processing and presentation system which provides data related to the reactions that take place within the test cartridge.

Although the description below refers to a sampling device, it may well be understood and therefore named as a collector, as it collects aerosol particles or particulate matter contained in a breath sample of a human or animal or in an environmental or room air sample. At the time the particles or particulate matter are collected, they are only suspected to contain respiratory pathogens and/or related biomarkers. Tests carried out thereafter will provide qualitative positive or negative confirmation or quantification of the pathogen or pathogens. Hence, the sampling device serves the purpose to sample respiratory pathogens and/or biomarkers possibly existing in the collected particles or particulate matter.

The description below also refers to an elution device that contains an aqueous solution suitable for dissolving an aqueous solution dissolvable element. It is conceivable that the aqueous solution dissolvable element and/or the filter element are located on a filter holder and/or support meshes that are not dissolvable by the aqueous solution. Hence, the particles which originate from the aerosol and which are sorbed by the aqueous solution dissolvable element and filtered by the filter element are washed out or eluted from the substrate by dissolution of the aqueous solution dissolvable element and/or eluting particles from the filter element and or full or partial dissolution of the filter element. Sorbtion by the aqueous solution dissolvable element and/or filter may include adsorption and/or absorption and/or binding and/or filtering.

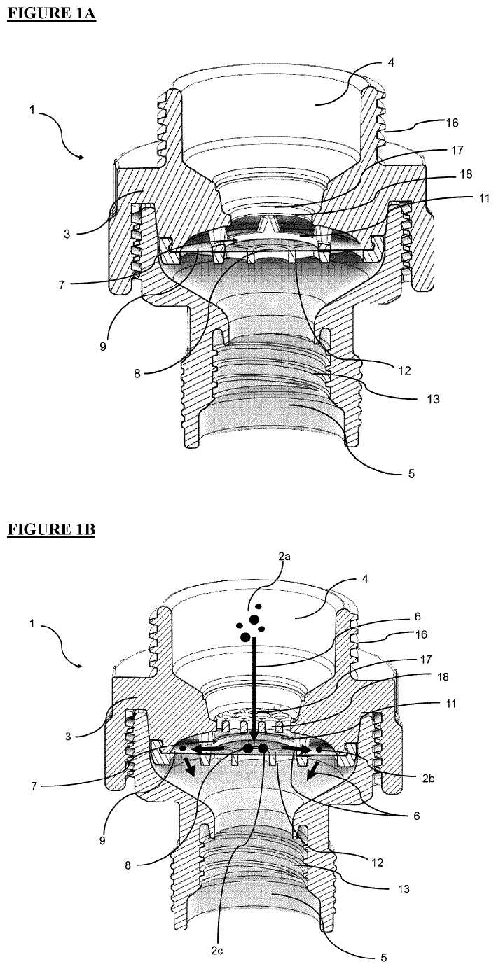

FIG. 1A-B show exemplary, non-limiting embodiments of the sampling device. Components with the same numbers in FIGS. 1A and 1B have the same or at least similar function. The sampling device 1 of FIG. 1A-B comprises a tubular housing 3 having a flow inlet 4 at one end and a flow outlet 5 at the other end opposite the flow inlet 4. Aerosol particles 2 flow along an airflow path 6 from the flow inlet 4 towards the flow outlet 5 and are captured inside the filter assembly 7 with the aqueous solution dissolvable element 8 and with the filter element 9. The tubular housing 3 is preferably of a two-piece design. The two parts are held together by means of a threaded or clamped engagement of the two parts. To this end, an external thread provided at the upper end of the lower part engages an internal thread provided at the lower end of the upper part.

An aqueous solution dissolvable element 8 and a ring-shaped filter element 9 are supported by a filter holder 12 which extends into tubular housing 3 in a perpendicular plane. Upstream of the aqueous solution dissolvable element 8 is provided an accelerator 17. The accelerator 17 preferably includes one or a plurality of nozzles 18 which act as an accelerator 17 as the nozzles 18 accelerate the aerosol particles 2a as they flow through the nozzles 18. The nozzles 18 of the accelerator 17 are preferably equidistantly spaced apart from each other across the inner diameter of the tubular housing 3. Preferably the sum of the area of all nozzle openings in the accelerator 17 is in the range from 20 to 200 mm².

The aqueous solution dissolvable element 8 and the filter element 9 are held in position by a retainer element 11. The retainer element 11, which is preferably formed as a one-piece part with the accelerator 17, and snaps into the filter holder 12 to clamp the aqueous solution dissolvable element 8 and the filter element 9 to keep them in place.

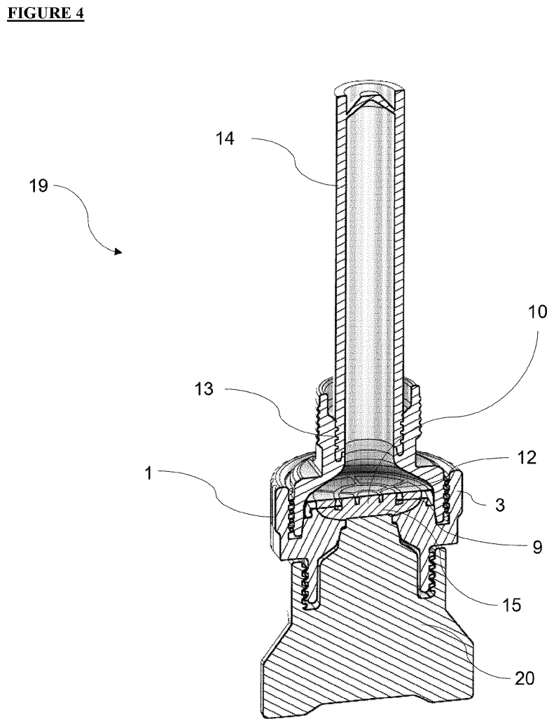

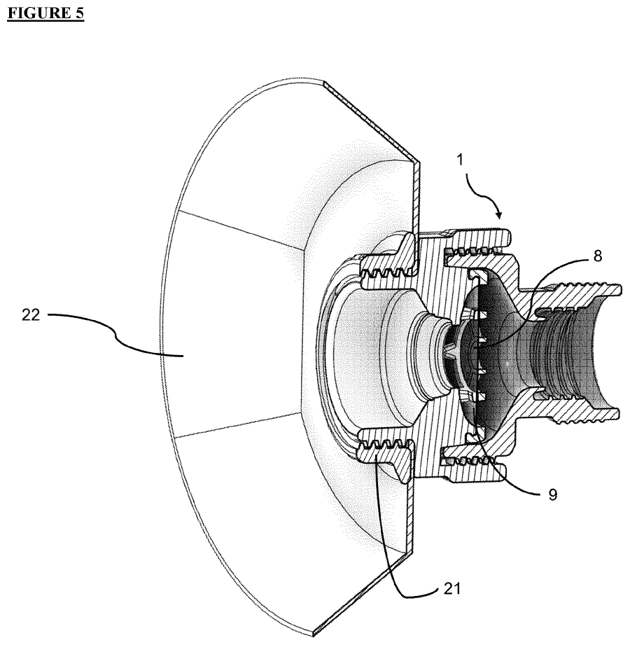

Upstream of the accelerator 17 is provided a thread 16 which may be used for the purpose of engaging a closure 20 (shown in FIG. 4) with the thread to close off the flow inlet 4. The thread 16 may be an internal or external thread depending on the type of closure. For example, the thread 16 may be an external thread provided on the inner surface of the housing 3 to introduce a threaded plug or lid to close off the flow inlet 4. The same thread 16 might be used to optionally connect the sampling device to a face mask 22 using a clamping ring 21 for long sampling times as illustrated in FIG. 5.

Another thread 13 is provided downstream of the aqueous solution dissolvable element 8 and the filter element 9 which may be used to screw into the tubular housing 3 an elution device 14 containing aqueous solution 15 to dissolve the aqueous solution dissolvable element 8 and eluate the collected aerosol particles on said aqueous solution dissolvable element 8 or on said filter element 9 as shown in FIG. 4.

As is also shown in FIG. 4, the elution device 14 is a tube with a closed end and an open end opposite the closed end. An external thread is provided in proximity of the open end of the elution device 14, which may be brought into engagement with the internal thread 13 provided downstream of the aqueous solution dissolvable element 8 that dissolves upon contact with the aqueous solution to build an opening 10 inside the sampling device 1.

It is conceivable to close off the flow outlet 5 by engaging a closure with the thread 13, and to use thread 16 for engagement with an elution device 14.

Instead of a thread 13, the sampling device 1 may well be provided with any type of engagement means to releasably engage the elution device 14 with the housing 3 of the sampling device 1. Such engagement means may be a clamping means, piercing means, such as the needle, a threaded engagement means, such as shown in FIG. 1, or other types of connecting or fastening means that are suitable to releasably engage the elution device 14 with the tubular housing 3 of the sampling device 1 in order to provide a fluidic communication pathway between the elution device 14 and the interior of the housing 3 of the sampling device 1.

The same or similar considerations may be applied to the thread 16.

The sampling device 1 shown in FIG. 1 and FIG. 4 is optimized towards efficient collection of pathogens present in human breath aerosol particles with low breathing resistance and high aerosol particle filter efficiency and subsequent dissolution of the aqueous solution dissolvable element 8 followed by complete elution of the aerosol particles from filter element 9 through submerging said filter element 9 due to formation of an opening 10 and subsequent easy collection of the sample in an elution device 14 such as a standard lab tube.

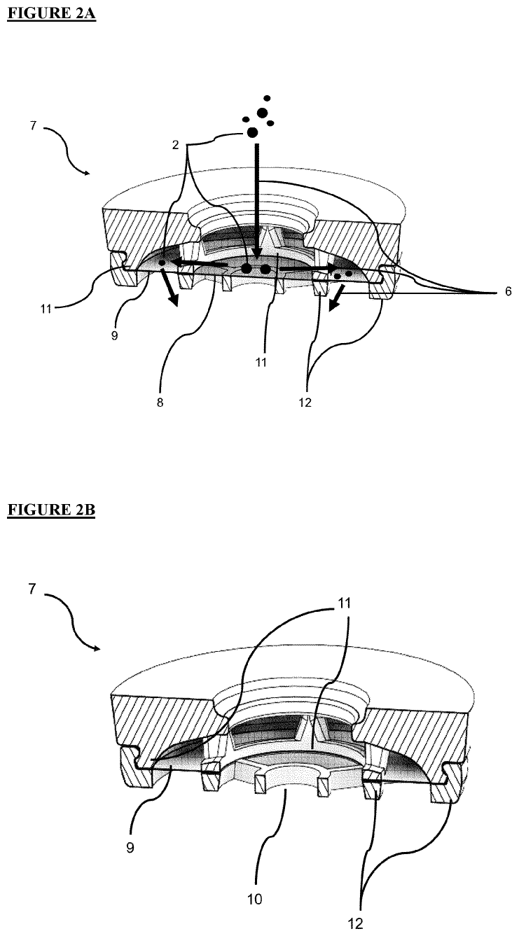

FIG. 2A-B show details of the filter assembly 7 of FIG. 1A in a perspective, cut-away side elevation. FIG. 2A illustrates the filter assembly 7 during collection of aerosol particles 2 and/or molecules and/or biomarkers and/or pathogens. Without wishing to be limited by a single hypothesis, the sampling device is designed to collect such molecules and/or structures and/or biomarkers and/or pathogens present in human breath aerosol particles. Aerosol containing aerosol particles 2 passes through flow path 6. When aerosol passes through flow path 6, non-gaseous components of the breath are concentrated at the aqueous solution dissolvable element 8. The dissolvable material 8 preferably comprises a aqueous solution dissolvable element which is held in position by a retainer element 11. These non-gaseous components comprise solid and liquid particles and build an aerosol together with the gaseous components. The aerosol can be a breath sample from a human or animal or an environmental sample or the like. If a respiratory pathogen is present in the lungs, throat, mouth and/or other parts of the respiratory tract, molecules and/or structures and/or biomarkers from such a pathogen or the entire pathogen itself are expected to be present in the aerosol particles 2. The aqueous solution dissolvable element 8 preferably comprises an airtight material through which such particles cannot pass, such as a foil, membrane, film, or the like such that the larger particles of the aerosol become trapped or are deposited on said dissolvable material 8 due to aerosol particles 2 inertia or interception. This forms a first aerosol particle collection stage. This first aerosol particle collection stage with the aqueous solution dissolvable element 8 can help to preserve the filter capacity of the second aerosol particle 2 collection stage by removing larger aerosol particles before the aerosol reaches the filter element 9. In a preferred embodiment the filter element 9 is ring-shaped and arranged radially outwardly from the aqueous solution dissolvable element 8. However other spatially close arrangements of the aqueous solution dissolvable element 8 and the filter element are possible. Alternative filter element embodiments include cylindric, conical filters or the like.

Both, the filter element 9 and the aqueous solution dissolvable element 8 are kept in place by a retainer element 11 that is clampingly engageable with a filter holder 12. In a preferred embodiment the filter element 9 and the aqueous solution dissolvable element 8 partially overlap to ensure that the entire sample is forced to pass through said filter element 9. In an alternative embodiment the filter element 9 and the aqueous solution dissolvable element 8 are bonded at the intersection with methods like ultrasonic bonding or the like. Without wishing to be limited by a single hypothesis, the filter assembly 7 is configured to support collection of such particles in a sufficiently concentrated amount with high filter efficiency, without requiring excessive exhalation force on the part of the subject or low backpressure if a pump is used to sample an environmental aerosol. Subjects having a reduced lung capacity, lung inflammation and/or otherwise unable to produce a great deal of force upon exhalation are able to provide a sufficient sample, due to the concentrating effect of the two collection stages combining the aqueous solution dissolvable element 8 and the filter element 9. The aqueous solution dissolvable element 8 and the filter element 9 are supported by the filter holder 12. The filter holder 12 is configured such that a large enough surface of the aqueous solution dissolvable element 8 is exposed to allow for dissolution of the aqueous solution dissolvable element 8 when brought into contact with an aqueous solution provided by the elution device 14 to form an opening 10 which results in submersion of the filter element 9 in aqueous solution. For that purpose, once the elution device 14 is screwed into the sampling device 1, the sampling device 1 together with the elution device 14 is turned upside down, thus causing the aqueous solution to flow out of the elution device 14, into the sampling device 1 and into contact with the aqueous solution dissolvable element 8 by effect of gravitation via the openings in the filter holder 12.

A variety of dissolvable polymer materials are available and suitable for use for the aqueous solution dissolvable element 8. Non-limiting examples of suitable dissolvable polymer materials include polyvinyl alcohol (PVA), chitosan, polyethylene oxide (PEO), pullulan, polyvinylpyrrolidone (PVP), polyvinyl acrylic acid (PVAc), poly methacrylic acid (PMAc), methyl acrylate copolymers, hydroxypropyl methylcellulose phthalate (HPMCP), or a combination thereof. Preferably the aqueous solution dissolvable element 8 is a membrane, foil or film. Preferably, the aqueous solution aqueous solution dissolvable element 8 used in the sampling device 1 described herein includes a PVA foil.

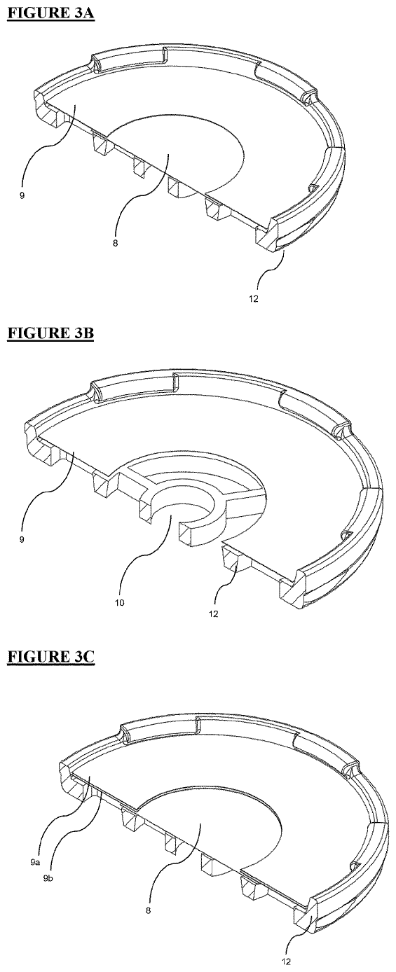

FIG. 3A-C show details of the filter assembly 7 from FIG. 1 and FIG. 2. FIG. 3A shows the arrangement of the filter element 9 and the aqueous solution dissolvable element 8 on the filter holder 12 with their partial overlap. FIG. 3B shows the centric opening that forms upon dissolution of the aqueous solution dissolvable element 8 upon elution. FIG. 3C illustrates a preferred embodiment of two stacked filters. A variety of filters are available and suitable for use for the filter element 9. Non-limiting examples of suitable filters include filters based on nonwoven fibers such as nanofiber mats or melt-blown fibers, woven materials, fiberglass, electrostatic filters, filter membranes or the like. The filter element can be non-dissolvable, partially or fully dissolvable in aqueous solution. Non-limiting examples for fiber production include electrospinning, melt blowing, blow spinning, wet spinning, direct drawing, centrifugal spinning, force spinning, touch- and brush-spinning, template synthesis, self-assembly, isolating fibers from plants or wood. In a preferred embodiment the filter element is a two-layer arrangement of two non-dissolvable polymer nanofiber mats on polymer support meshes having a thickness in the range between 40 μm to 500 μm to support the fibers. Preferably the support meshes are a poly(hexamethylene adipamide) (PA 6.6) support mesh (such as Sefar Nitex 03-130/52, Sefar AG, Switzerland). In a preferred embodiment the fibers have a dimeter in the range between 20 nm to 1 μm, preferably 300 nm. The stacking of two filters (FIG. 3C) has the advantage that the upper filter 9a can compensate small defects or inconsistencies of the lower filter 9b and vice versa to achieve high aerosol particle filter efficiencies at low pressure resistance.

In some embodiments the aqueous solution dissolvable element 8 may contain agents or detergents to increase stability during collection and improve solubility during elution. Non-limiting examples include Triton X-100, Tween, SDS, dichlorodiphenyltrichloroethane (DDT), chaotropic salts, Dithiothreitol (DTT), acids and/or bases, pH buffer salts, beads, or any combinations thereof.

The aqueous solution 15 that is introduced with the elution device 14 may contain lysing agents or detergents and may be configured to create a lysate. Non-limiting examples of lysing agents or detergents include Triton X-100, Tween, SDS, dichlorodiphenyltrichloroethane (DDT), chaotropic salts, Dithiothreitol (DTT), acids and/or bases, pH buffers, beads, solvents, or any combinations thereof. In a preferred embodiment the aqueous solution is a TRIS-EDTA buffer with a pH above 8.0 and directly compatible with nucleic acid amplification tests (NAATs) such as PCR. By way of example the tube containing the aqueous solution 15 can be custom made or be a standard centralized laboratory tube containing PCR transport media such as tubes from the cobas PCR Media kit (Roche Diagnostics, Switzerland) or the universal viral transport (UVT) system (Becton Dickinson, USA) or MSwab (Copan, Italy). In a preferred embodiment the elution device 14 contains 2 ml of aqueous solution.

FIG. 4 shows an exemplary, non-limiting embodiments of the sampling device 1, the elution device 14 and closure 20 such as a lid or plug. By way of example the sampling device 1 can also be transported and/or stored together with the elution device 14 with the aqueous solution 15 as the aqueous solution may help to stabilize the pathogen and/or biomarkers during storage and/or transportation. The aqueous solution can also inactivate the pathogen for safe transportation and storage.

### Example 1—Evaluation of the Sampling Device for the Collection of Mycobacterium from Simulated Breath Aerosols with Elution and Detection Using PCR

This non-limiting Example relates to an optimized illustrative sampling device for the collection of Mycobacterium aerosol particles with elution using the elution device and subsequent detection using polymerase chain reaction (PCR).

### Materials and Methods

### Aqueous Solution Dissolvable Element

Commercially available polyvinyl alcohol (PVA) film with a thickness of 35 μm was punched with a handle punch with a diameter of 15 mm

### Filter Element

Polyacrylonitrile (PAN) nanofibers were spun onto a PA6.6 support mesh (Sefar Nitex 03-130/52, Sefar AG, Switzerland) using free surface electrospinning with a Nanospider NS Lab 500 (Elmarco, Czech Republic). The resulting filter mat was punched with two handle punches to give a ring-shaped filter element with 11 mm inner and 25 mm outer diameter.

### Sampling Device

The Selective Laser Sintering (SLS) 3D printed custom made sampling device housing out of polyamide 12 (PA2200, EOS GmbH, Germany) had an outer diameter of 38 mm and a length of 45 mm. the sampling device housing was washed an autoclaved. The PVA film and two stacked filter elements were positioned on the filter holder and fixed with the retainer element. The sampling device design is illustrated in FIG. 1A, the filter assembly in FIG. 2, and the details of the filter assembly in FIG. 3C.

### Simulated Mycobacterial Aerosols

Mycobacterium bovis BCG stocks (7.19×10⁶ cfu/ml) were serially diluted in 1×TE buffer pH 8 (10 mM Tris, 1 mM EDTA) to achieve 10, 50, 100, and 500 cfu per 70 μl. The TE buffer but without BCG was used to generate “negative” samples. Approximately 70 μl of these BCG suspensions with different cell concentrations or the negative sample were nebulized with a Cirrus2 nebulizer (Intersurgical, UK) using HEPA-filtered air at 6 l/min to achieve BCG inputs of 0 (“negative”), 10 cfu, 50 cfu, 100 cfu, and 500 cfu in the aerosol. The nebulizer outlet was connected to a T-piece to dilute the aerosol with 34 l/min HEPA-filtered air to achieve a total mass flow of 40 l/min. Air flow rates were controlled with two SFC5500 mass flow controllers (Sensirion, Switzerland) controlled by a custom-made Lab View software (National Instruments, Switzerland). The output of the T-piece with the diluted aerosol was connected to the inlet of the sampling device for 15 seconds of aerosol sampling per experiment. 6 repeats per concentration level (0, 10, 50, 100, 500 cfu BCG) were performed with the sampling device. In addition 6 repeats were performed with the sampling device after exposure to 20 human breaths of a healthy subject at the 500 cfu BCG level to assess the stability of the sampling device after exposure to human breath.

### Elution

After the collection of the aerosol particles, an elution device which is a standard laboratory tube containing 2 ml of 1×TE buffer pH 8 (10 mM Tris, 1 mM EDTA) was connected to the sampling device via the thread on the outlet side. The sampling device was closed with a plug via the thread on the inlet side. After closing of the sampling device with the laboratory tube and plug, the arrangement was turned upside-down for 4 minutes so that the TE buffer comes into contact with the aqueous solution dissolvable element to form an opening upon dissolution to elute the collected BCG bacteria from the filter element and the dissolvable material. After turning the arrangement again, the eluate runs into the elution device/tube and the sampling device was removed. The eluate in the tube was used for subsequent analysis with PCR.

### DNA Extraction and PCR Detection

DNA was extracted from the entire eluate of approximately 1.8 ml in the tube by using a spin column protocol of the Qiagen QIAamp DNA minikit (Qiagen, Switzerland) as described in the handbook. 9 μL of extracted DNA was subsequently used for PCR on a QuantStudio5 qPCR cycler (ThermoFisher, Switzerland) to amplify IS6110, a gene unique to M. tuberculosis complex.

### Analysis

Ct values of PCR positive samples were plotted by cfu concentration. A probit model according to CLSI EP14A guideline was used to estimate the limit of detection (LOD).

### Results

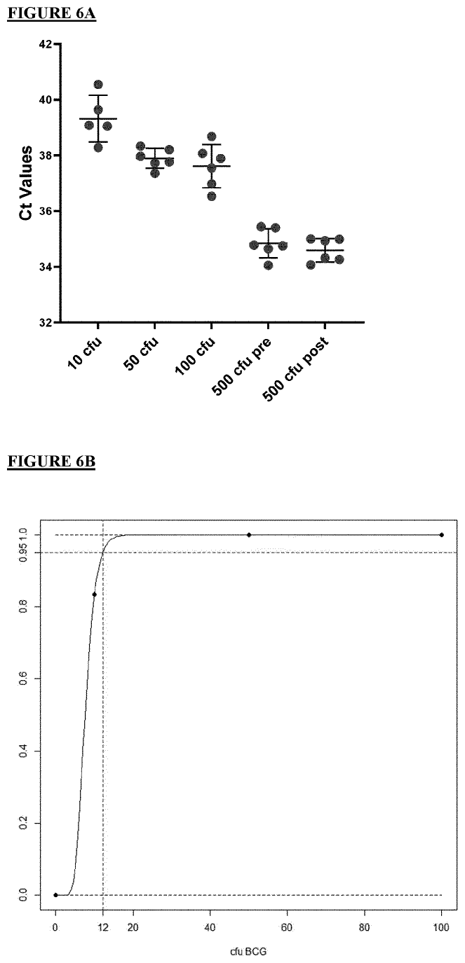

Table 1 shows the results of 36 aerosol experiments for the sampling device and elution device in combination with the detection using PCR. FIG. 6A shows PCR Ct values of all positive samples and a clear trend towards longer Ct values for aerosol samples with lower BCG input. There was no significant difference in the Ct's of collectors pre- and post-exposure to human breath suggesting that the sampling device is sufficiently stable during collection. Table 1 shows the probability of positivity per cfu concentration level. The negative aerosol collected with the sampling device and eluted with the elution device remained negative for all samples (0/6). The PCR delivered positive results for 83% (5/6) of aerosol samples with an input of 10 cfu BCG and 100% (6/6) of aerosol samples with an input of 50 cfu BCG and above, suggesting a limit-of-detection (LOD) of the sampling device in combination with the elution device and the DNA extraction and PCR detection between 10 to 50 cfu BCG per aerosol sample. An LOD of 12 cfu BCG was determined using the probit model based on binary logistic regression fitted through the tested concentration with the LOD defined as the cfu BCG input where the probability of sample positivity reaches 95%. The probit curve is shown in FIG. 6B.

TABLE 1

| | Positive samples/total | |
|---|---|---|
| Input BCG (cfu) | number of samples | Probability of positivity |
| 0 | 0/6 | 0% |
| 10 | 5/6 | 83% |
| 50 | 6/6 | 100% |
| 100 | 6/6 | 100% |
| 500 | 6/6 | 100% |
| 500 post-breath | 6/6 | 100% |

### Example 2—Evaluation the Aerosol Particle Filter Efficiency of the Sampling Device from Example 1

This non-limiting Example relates to an illustrative sampling device from Example 1 for the collection of aerosol particles.

### Materials and Methods

The sampling device was produced as described in Example 1 and is illustrated in FIG. 1A. For this example the sampling device is integrated into a face mask as illustrated in FIG. 5. Filter efficiency was tested according to European Norm EN 13274-7 using a PMFT 1000 test system (Palas, Germany) using oil aerosol particles and a Promo 1000 aerosol photometer (Palas, Germany) for the measurement of aerosol size distribution.

### Results

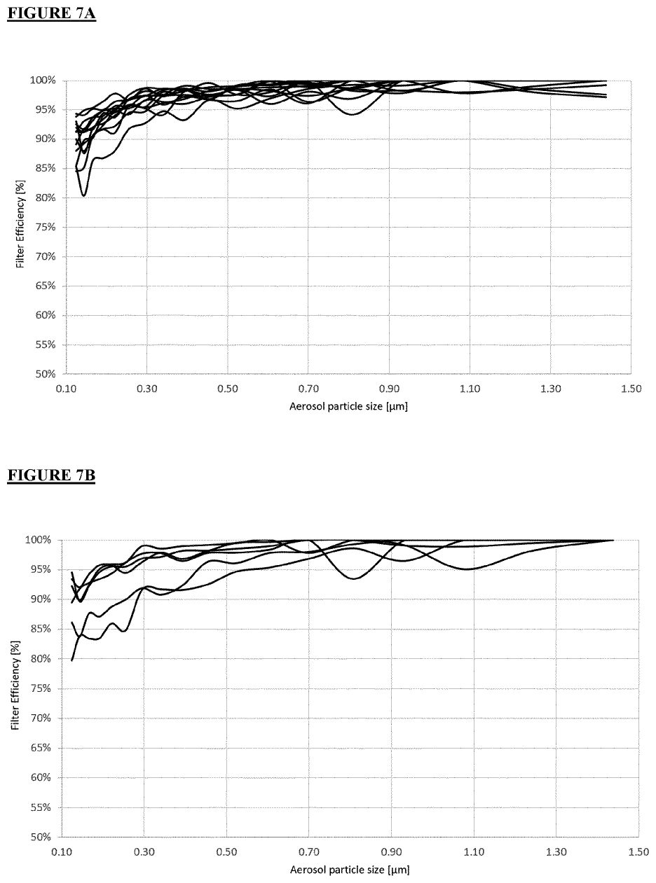

FIG. 7A shows filter efficiency results of 13 independent filter efficiency experiments with the sampling devices integrated in FFP2 face masks for aerosol sizes between 100 nm and 1.44 μm. Filter efficiency is consistently above 90% for aerosol sizes above or equal to 300 nm. The cumulative filter efficiency in the aerosol size range between 300 nm and 2.2 μm is 97.4% (95% Confidence Interval 96.7-98.1%).

FIG. 7B shows results of six independent filter efficiency experiments of the sampling devices integrated in FFP2 face masks post exposure to 20 human exhalations into the sampling device. It shows that filter efficiency remains above 90% post exposure to human exhalate suggesting that the sampling devices maintain their filter efficiency after exposure to human breath. The cumulative filter efficiency post exposure to human breath in the aerosol size range between 300 nm and 2.2 μm is 97.0% (95% CI: 95.5-98.5%).

### Example 3—Breathing Resistance of the Sampling Device from Example 1

This non-limiting Example relates to an illustrative sampling device from Example 1.

### Materials and Methods

### Pressure Drop Measurement

Breathing resistance of three independent sampling devices from Example 1 were measured by determining the pressure drop at different air mass flow rates between 10 and 80 l/min. Pressure drop over the sampling device was measured using a PREMASGARD 1115-I LCD (S+S Regeltechnik, Germany) differential pressure sensor. Air flow rates were controlled with a SFC5500 mass flow controller (Sensirion, Switzerland).

### Results

### Pressure Drop Measurement

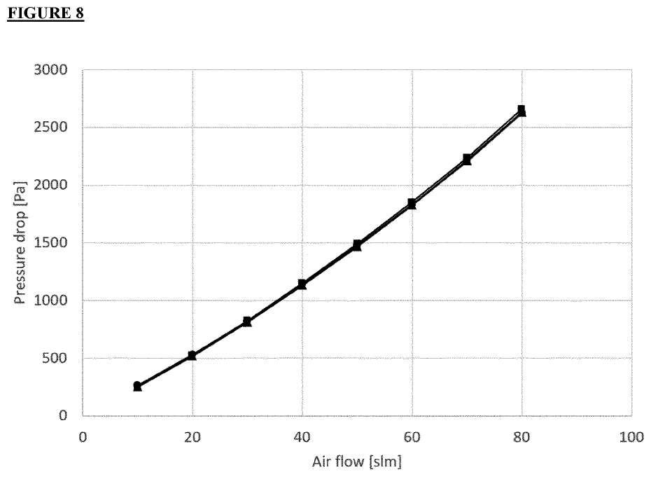

FIG. 8 shows the pressure drops from three sampling devices at different air mass flow rates. The average differential pressure (or breathing resistance) of the sampling device at 40 l/min was 1142 Pa. The average differential pressure of the sampling device at 50 l/min was 1481 Pa. The average differential pressure of the sampling device at 60 l/min was 1837 Pa. This breathing resistance is sufficiently low to collect exhaled breath without exposing the patient to stress.

1. A sampling device (1) for the collection of aerosol particles (2) contained in an aerosol sample, the sampling device (1) comprising:
a housing (3) having a flow inlet (4) for the inflow of an aerosol sample into the housing (3) and a flow outlet (5) for the outflow of the aerosol sample out of the housing (3), the flow of the aerosol sample from the flow inlet (4) to the flow outlet (5) defining a flow path (6) of the aerosol sample, and
a filter assembly (7) to collect aerosol particles (2) contained in the aerosol sample,
wherein the filter assembly (7) includes an aqueous solution dissolvable element (8) and a filter element (9), and wherein the filter assembly (7) is at least partially arranged inside the housing (3) between the flow inlet (4) and the flow outlet (5) and in the flow path (6) of the aerosol sample.

2. The sampling device (1) of claim 1, wherein both the aqueous solution dissolvable element (8) and the filter element (9) are arranged inside the housing (3) between the flow inlet (4) and the flow outlet (5) and in the flow path (6) of the aerosol sample, and are thus both exposed to the aerosol sample.

3. The sampling device (1) of claim 1, wherein the aqueous solution dissolvable element (8) is airtight and wherein the filter element (9) is air permeable.

4. The sampling device (1) of claim 1, wherein an area of the filter element (9) is greater than an area of the aqueous solution dissolvable element (8).

5. The sampling device (1) of claim 1, wherein the filter element (9) is at least partially arranged radially outwardly from the aqueous solution dissolvable element (8).

6. The sampling device (1) of claim 1, wherein the filter element (9) is ring-shaped and/or circular, and the aqueous solution dissolvable element (8) is geometrically arranged completely inside the filter element (9).

7. (canceled)

8. (canceled)

9. (canceled)

10. The sampling device (1) of claim 1, wherein the filter assembly (7) further includes a retainer element (11) which retains the filter element (9) and/or the aqueous solution dissolvable element (8) in place.

11. The sampling device (1) of claim 10, wherein the retainer element (11) is clampingly engageable with a filter holder (12).

12. The sampling device (1) of claim 1, further comprising an engagement means (13) for releasably engaging an elution device (14) with the housing (3), for introducing an aqueous solution (15) into the housing (3), for submerging the aqueous solution dissolvable element (8) and the filter element (9) into the aqueous solution (15), and to thus dissolve the aqueous solution dissolvable element (8) upon contact with an aqueous solution (15).

13. The sampling device (1) of claim 12, wherein the engagement means (13) includes a thread.

14. (canceled)

15. The sampling device (1) of claim 13, wherein the engagement means (13) is configured such that a laboratory tube (14) containing an aqueous solution (15) and normally used in preparation for an assay selected from the group consisting of a nucleic acid amplification test (NAAT), a PCR assay, an isothermal amplification assay, a DNA hybridization assay, a CRISPR-based assay, a sequencing assay and an immunoassay, is releasably connectable to the housing (3).

16. The sampling device (1) of claim 1, wherein a thread (16) is provided on the housing (3) upstream of the aqueous solution dissolvable element (8), and wherein the thread (16) is engageable with a threaded closure (20) to close off the flow inlet (4) of the housing (3).

17. The sampling device (1) of claim 1, wherein the aqueous solution dissolvable element (8) comprises a polymer selected from the group consisting of polyvinyl alcohol (PVA), chitosan, polyethylene oxide (PEO), pullulan, polyvinylpyrrolidone (PVP), polyvinyl acrylic acid (PVAc), poly methacrylic acid (PMAc), methyl acrylate copolymers, hydroxypropyl methylcellulose phthalate (HPMCP), and a combination thereof.

18. (canceled)

19. The sampling device (1) of claim 1, wherein the filter element (9) is fully or partially dissolvable in an aqueous solution (15).

20. The sampling device (1) of claim 1, wherein the filter element (9) comprises fibers.

21. (canceled)

22. (canceled)

23. The sampling device (1) of claim 20, wherein the filter element (9) further comprises a polymer support mesh having a thickness in a range between 40 μm to 500 μm to support the fibers.

24. The sampling device (1) of claim 1, wherein dissolution of the aqueous solution dissolvable element (8) and/or the filter element (9) in the aqueous solution (15) is triggered by a change in pH-value.

25. The sampling device (1) of claim 1, wherein the aqueous solution dissolvable element (8) and/or filter element (9) contain assay reagents selected from the group consisting of control nucleic acid, primers, probes, nucleotides, enzymes, salts, capture beads and combinations thereof.

26. (canceled)

27. A system (19) for the collection and elution of aerosol particles contained in an aerosol sample, the system (19) comprising:
a sampling device (1) of claim 1, and
an elution device (14) releasably connectable to the housing (3) by means of the engagement means (13) of the sampling device (1) for introducing an aqueous solution (15) into the housing (3), for dissolving the aqueous solution dissolvable element (8) upon contact with the aqueous solution (15), and upon dissolution of the aqueous solution dissolvable element (8), for submerging the filter element (9) into the aqueous solution (15) to thus retrieve the aerosol particles (2) captured on the aqueous solution dissolvable element (8) and the filter element (9) and receiving the eluate.

28. The system (19) of claim 27, wherein the elution device (14) is a tube having a closed end and an open end opposite the closed end, and wherein an external thread is provided at the open end of the elution device which is engageable with an internal thread (13) of the housing (3) of the sampling device (1).

29. (canceled)
